(19) United States
(12) Patent Application Publication
(10) Pub. No.: US 2022/0249540 A1
(43) Pub. Date: Aug. 11, 2022

(54) INHALABLE COMPOSITION COMPROMISING CYCLODEXTRIN FOR USE IN THE TREATMENT OF NASAL INFLAMMATIONS

(71) Applicant: AQUILON PHARMACEUTICALS SA, Vise (BE)

(72) Inventors: DIDIER CATALDO, OLNE (BE); MARIE-ASTRID ALBERT, EMBOURG (BE); PAUL MAES, VISE (BE)

(21) Appl. No.: 17/623,554

(22) PCT Filed: Jun. 30, 2020
(86) PCT No.: PCT/EP2020/068355

§ 371 Date: Dec. 28, 2021

(30) Foreign Application Priority Data
Jul. 1, 2019 (BE) 2019/5421

(51) Int. Cl. A61K 31/724; A61K 47/02; A61K 9/00; A61P 11/02

(52) U.S. Cl. CPC A61K 31/724; A61K 47/02; A61K 9/0075; A61P 11/02; A61K 9/0043

(57) ABSTRACT

The present invention relates to an inhalable composition comprising cyclodextrin for the treatment of nasal inflammations. In a preferred embodiment, the treatment is topical.

### TECHNICAL DOMAIN

The present invention relates to an inhalable isotonic composition containing cyclodextrins or derivatives thereof for the treatment of nasal inflammations.

### TECHNOLOGICAL BACKGROUND OF THE INVENTION

The use of cyclodextrins as an excipient or drug-delivery-system is known. Typically, cyclodextrins are used to enhance delivery of other compounds and in particular of active pharmaceutical ingredients.

Also, aqueous compositions containing cyclodextrin as a solubilizing excipient for the treatment of diseases are known. Cyclodextrins are thereby said to produce very little or no side effects whilst substantially increasing the solubility of active pharmaceutical ingredients in water. In particular cyclodextrins are used to solubilize water-insoluble or less-soluble active pharmaceutical ingredients and more particular anti-inflammatory active pharmaceutical ingredients.

U.S. Pat. No. 7,029,657B2 describes administering intranasally an estrogenic compound and an androgenic compound complexed with a cyclodextrin as an intranasal drug-delivery system for use in contraception or in treatment of benign gynecological disorders.

EP3151836 to the University of Liège and Paul Maes discloses the use of cyclodextrin in conjunction with the corticosteroid budesonide for the treatment and prevention of bronchial inflammatory diseases.

EP1799231 to the University of Liège proposes the direct administration of cyclodextrins for the treatment of bronchial inflammatory disease.

Recently, WO2019067269 to Asdera LLC describes the use of cyclodextrins alone or in combination with a therapeutic compound in diseases and disorders involving phospholipid dysregulation.

Document WO2006000421 to Wacker describes the use of alpha-cyclodextrins as a food for the treatment of allergic nasal inflammations. This document does not disclose the topical administration by inhalation nor the particle size distribution of the droplets or microparticles.

However, the use of cyclodextrins for the intranasal treatment of nasal inflammations, such as sinusitis or rhinitis is not disclosed in any of the aforementioned publications.

In recent years, the number of nasal inflammations has substantially increased through multiple factors such as decreasing air quality. Examples of nasal inflammations are diseases like rhinitis or sinusitis. However, many of the treatments cause either side effects or lack efficiency.

There is therefore a general need to provide alternative, better and cost-effective intranasal treatments of nasal inflammations with a minimum of side effects.

### BRIEF SUMMARY OF THE INVENTION

The present inventors surprisingly have found that the intranasal administration of cyclodextrins may directly and substantially reduce nasal mucosa secretion when administered in effective amounts. In particular, the cyclodextrins may be used for the intranasal treatment or prevention of nasal inflammations without the need for a further active ingredient. In particular, the present inventors have surprisingly found that the number of goblet cells in nasal mucosa tissue may be reduced through the administration of an inhalable composition comprising cyclodextrins without the need for an active pharmaceutical ingredient for which cyclodextrins are used as excipients to increase solubility in water or as a drug delivery system and in particular without the need for anti-inflammatory active pharmaceutical ingredients.

Accordingly, the present invention is directed at an inhalable composition comprising one or more cyclodextrins or pharmaceutically acceptable derivatives thereof for use in the prevention or treatment of nasal inflammations.

In another embodiment, the present invention is directed at an inhalable composition, wherein the nasal inflammation is a viral nasal inflammation.

In another embodiment, the present invention is directed at an inhalable composition comprising one or more cyclodextrins or pharmaceutically acceptable derivatives thereof for use in the prevention or treatment of nasal inflammations by topical treatment of the nose, and in particular of the nasal mucosa, for example in the form of a topical nasal spray. Preferably, the topical nasal composition or spray of the invention does not primarily target any other tissue or part of the body than the nose or the nasal mucosa.

In another embodiment, the present invention is directed at an inhalable topical spray comprising one or more cyclodextrins or pharmaceutically acceptable derivatives thereof for use in the prevention or treatment of nasal inflammations.

In another embodiment, the inhalable composition of the invention, preferably a topical nasal spray, is characterized by the fact that no further active pharmaceutical ingredient is present in therapeutically effective amounts for which cyclodextrin is used as a drug-delivery system for other parts of the body or tissues than the nose or the nasal mucosa.

In another embodiment, the inhalable composition of the invention, preferably a topical nasal spray, is characterized by the fact that no further active pharmaceutical ingredient is present in therapeutically effective amounts for which cyclodextrin is used to increase its solubility in water.

In another embodiment, the inhalable composition of the invention, preferably a topical nasal spray, is characterized by the fact that no further anti-inflammatory active pharmaceutical ingredient is present in therapeutically effective amounts.

In another embodiment, the present invention is directed at an inhalable composition comprising one or more cyclodextrins or pharmaceutically acceptable derivatives thereof for use in the prevention or treatment of nasal inflammations, wherein no further active pharmaceutical ingredient is present in therapeutically effective amounts for which cyclodextrin is used to increase its solubility in water.

In another embodiment, the inhalable composition comprises no further anti-inflammatory active pharmaceutical ingredient in therapeutically effective amounts.

Moreover, the present invention is directed at an inhalable aqueous composition comprising one or more cyclodextrins or pharmaceutically acceptable derivatives thereof for use in the prevention or treatment of nasal inflammations, wherein no further active pharmaceutical ingredient is present in therapeutically effective amounts.

Another aspect of the invention is an inhalable composition, wherein the inhalable composition is an aerosolizable aqueous composition, preferably a topical nasal spray, or an inhalable dry powder composition.

Another aspect of the invention is an inhalable composition, wherein the pH is from 3.5 to 7.5.

Another aspect of the invention is an inhalable composition, wherein the pH is from 6.5 to 7.

Another aspect of the invention is an inhalable composition, wherein the cyclodextrin is selected from the group consisting of alpha-cyclodextrin, beta-cyclodextrins, gamma-cyclodextrin, 2-hydroxypropyl-betabeta-cyclodextrin, 2-hydroxypropyl-gamma-cyclodextrin, sulfobutylether-beta-cyclodextrin, and methyl-beta-cyclodextrin.

Another aspect of the invention is an inhalable composition, wherein the cyclodextrin is hydroxypropylbetacyclodextrin.

Another aspect of the invention is an inhalable composition, wherein no corticosteroid or oil is present in effective amounts.

Another aspect of the invention is an inhalable composition, wherein the inhalable composition is isotonic.

Another aspect of the invention is an inhalable composition, wherein the inhalable composition is hypertonic.

Another aspect of the invention is an inhalable composition wherein the concentration of cyclodextrins is from 1 mg/ml to 100 mg/ml.

Another aspect of the invention is an inhalable composition wherein the concentration of cyclodextrins is from 5 mg/ml to 50 mg/ml.

Another aspect of the invention is an inhalable composition wherein the concentration of cyclodextrins is from 10 mg/ml to 30 mg/ml.

Another aspect of the invention is an inhalable, in particular an inhalable aqueous composition wherein the concentration of cyclodextrins is from 0.5 mM (millimolar) to 100 mM, preferably 1 mM to 75 mM, even more preferably 2.5 mM to 60 mM, even more preferably 5 mM to 50 mM, even more preferably 10 mM to 40 mM, even more preferably 20 mM to 30 mM, even more preferably 40 millimolar to 60 mM, even more preferably 25 mM.

Another aspect of the invention is an inhalable composition, wherein the composition consists of:
a) 0.5 to 100 mM of one or more cyclodextrins or pharmaceutically acceptable derivatives thereof;
b) 0.1 to 5 mg/ml of a pharmaceutically acceptable salt selected from the group consisting of pH buffers, sea salt, and salt and
c) water.

Another aspect of the invention is an inhalable composition, wherein the composition consists of:
a) 10 to 40 mM of one or more cyclodextrins or pharmaceutically acceptable derivatives thereof;
b) 0, 5 to 3 mg/ml of a pharmaceutically acceptable salt selected from the group consisting of pH buffers, sea salt, and salt, and
c) water.

Another aspect of the invention is an inhalable composition, wherein the composition consists of:
a) 20 to 30 mM of one or more cyclodextrins or pharmaceutically acceptable derivatives thereof;
b) 1 to 3 mg/ml of a pharmaceutically acceptable salt selected from the group consisting of pH buffers, sea salt, and salt, and
c) water.

Another aspect of the present invention is an inhalable composition, wherein the composition consists of:
a) 1 to 100 mg/ml volume of one or more cyclodextrins or pharmaceutically acceptable derivatives thereof;
b) 0.5 to 5 mg/ml of a pharmaceutically acceptable salt selected from the group consisting of pH buffers, sea salt, and salt and
c) water.

Another aspect of the present invention is an inhalable composition, wherein the composition consists of:
a) 5 to 50 mg/ml of one or more cyclodextrins or pharmaceutically acceptable derivatives thereof;
b) 1 to 3 mg/ml of a pharmaceutically acceptable salt selected from the group consisting of pH buffers, sea salt, and salt and
c) water.

Another aspect of the present invention is an inhalable composition, wherein the composition consists of:
a) 10 to 30 mg/ml of one or more cyclodextrins or pharmaceutically acceptable derivatives thereof;
b) 1 to 3 mg/ml of a pharmaceutically acceptable salt selected from the group consisting of pH buffers, sea salt, and salt and
c) water.

Another aspect of the invention is a method of prevention or treatment of nasal inflammations, wherein cyclodextrin is administered per inhalation in an amount effective to reduce or prevent the nasal inflammation, preferably without causing treatment limiting side effects, such as those selected from the group consisting of treatment limiting side effects, such as those selected from the group consisting of renal clearance, hepatic impairment as expressed by elevated levels of transaminase, and wheezing after administration, as compared to subjects untreated with the cyclodextrin.

In another aspect of the method of prevention or treatment of nasal inflammations, the cyclodextrin is administered per inhalation in the form of a 15 mM HPBCD saline isotonic solution.

In another aspect of the method of prevention or treatment of nasal inflammations, the cyclodextrin is administered per inhalation in the form of a 15 mMol HPBCD PBS ph7.4 based solution.

In another aspect of the method of prevention or treatment of nasal inflammations, the cyclodextrin is administered per inhalation in the form of a 5 mMol HPBCD saline isotonic solution.

In another aspect of the method of prevention or treatment of nasal inflammations, the cyclodextrin is administered per inhalation in the form of a 25 mMol HPBCD PBS ph7.4 based solution.

In another aspect of the method of prevention or treatment of nasal inflammations, the cyclodextrin is administered per inhalation in the form of a 40 mMol HPBCD saline isotonic solution.

In another aspect of the method of prevention or treatment of nasal inflammations, the cyclodextrin is administered per inhalation in the form of a 25 mMol HPBCD saline isotonic solution.

In another aspect of the method of prevention or treatment of nasal inflammations, the cyclodextrin is administered per inhalation in the form of a 10 mMol HPBCD citrate ph. 4.5 based solution.

In another aspect of the method of prevention or treatment of nasal inflammations, the cyclodextrin is administered per inhalation in the form of a 40 mMol HPBCD citrate ph. 4.5 based solution.

In another aspect of the method of prevention or treatment of nasal inflammations, the cyclodextrin is administered per inhalation in the form of a 50 mMol HPBCD saline isotonic solution.

Another aspect of the present invention is an aerosol generating device or a dry powder inhaler comprising the composition of the present invention.

Other embodiments according to the present invention are mentioned in the appended claims.

### DETAILED DESCRIPTION OF THE INVENTION

Other characteristics and advantages of the present invention will be derived from the non-limitative following description, and by making reference to the drawings and the examples.

The term “aqueous composition” as used herein refers to a composition comprising at least one cyclodextrin, water and optionally one or more other components suitable for use in pharmaceutical delivery such as carriers, stabilizers, diluents, dispersing agents, suspending agents, thickening agents, excipients, and the like. The disclosure relates to a pharmaceutical composition comprising an effective amount of any cyclodextrin molecule or derivative or salt disclosed herein and a pharmaceutically acceptable carrier.

In some embodiments, the pharmaceutical composition is free of alpha or gamma-cyclodextrin.

The term “topical” means that the composition of the invention is applied to the nose, in particular to the nasal mucosa, for example by intranasal application or through inhalation of an aerosol.

In the sense of the present invention “topical nasal” means a purpose-limitation in the sense that no other tissue or part of the body than the nose and in particular the nasal mucosa is targeted by the administration of the composition of the present invention.

The term “directly” in conjunction with the treatment described in the present invention means that the main or primary purpose of the cyclodextrins of the composition of the invention is the use for treatment or prevention of nasal inflammations. Consequently, in one embodiment, the main or primary purpose of the cyclodextrins is not to enhance or improve the delivery or solubility of another active pharmaceutical ingredient.

The term “active pharmaceutical” or “API” refers to any substance or combination of substances used in a finished pharmaceutical product, intended to furnish pharmacological activity or to otherwise have direct effect in the diagnosis, cure, mitigation, treatment or prevention of disease, or to have direct effect in restoring, correcting or modifying physiological functions in human beings. Preferably, the term “active pharmaceutical ingredient” refers to a molecule that is intended to be biologically active, for example for the purpose of treating inflammatory, autoimmune, or pulmonary disease, disorder, or condition. Even more preferably, the term “active pharmaceutical ingredient” refers to substances which whose placing on the market requires a marketing authorization, for example as foreseen under directive 2001/83/EC of 6 Nov. 2001 on the Community code relating to medicinal products for human use.

Typical active pharmaceutical ingredients include anti-inflammatory compounds and in particular corticosteroids which are commonly also referred to as steroids, corticoids, glucocorticoids, or cortisol analogues. Examples of such corticosteroids include beclomethasone, budesonide, flunisolide, fluticasone, ciclesonide, mometasone, or any compounds comprising the active moiety of any of these corticosteroids, such as salts, derivatives, and prodrugs thereof.

The term “treatment” or “treat” describes any treatment of nasal inflammation for example by inhalation of an aerosol or a micronized powder.

The terms “effective amount” or “therapeutically effective amount”, as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study. In some embodiments, as used herein, the term “therapeutically effective amount” is meant to refer to an amount of an active agent or combination of agents effective to ameliorate, delay, or prevent the symptoms. Determination of a therapeutically effective amount is well within the capabilities of those skilled in the art, especially in light of the detailed disclosure provided herein.

The term “nasal inflammation” describes a wide range of inflammatory diseases of the nose, for example sinusitis or rhinitis. These inflammations may be of viral origin, of bacterial origin or allergen-induced or any other inflammation. In a preferred embodiment, the nasal inflammation is a viral inflammation. In another embodiment, the nasal inflammation is caused by a virus selected from the group consisting of adenovirus, human bocavirus, human coronavirus, human metapneumovirus, human parainfluenza virus, human respiratory syncytial virus, human rhinovirus, pharyngoconjunctival fever, and any other virus causing severe acute respiratory syndrome or combinations thereof.

The inhalable composition of the invention primarily comprises a cyclodextrin. Cyclodextrins are oligosaccharides composed of glucopyranose units. The major unsubstituted cyclodextrins are usually prepared by the enzymatic degradation of starch.

The cyclodextrin of the invention may be any cyclodextrin, in particular alpha-, beta- and gamma-cyclodextrins, comprising 6, 7 and 8 glucopyranose units, respectively.

In another embodiment of the invention derivatives of cyclodextrins are used, for example chemically modified cyclodextrins, which may have increased water solubility over unmodified cyclodextrins. Examples of such derivatives include in particular 2-hydroxypropyl-beta-cyclodextrin (HPBCD), 2-hydroxypropyl-γ-cyclodextrin (HPGCD), sulfobutylether-beta-cyclodextrin (SBEBCD), and methyl-beta-cyclodextrin (MBCD).

In another embodiment, the amount or content of the cyclodextrin in the composition is selected to ensure a sufficiently low viscosity at ambient temperature in view of inhalation. The dynamic viscosity, which may also be influenced by the choice and quantity of the further excipients, also has a clear influence on the particle size distribution of the aerosol formed by nebulization and on the efficiency of nebulization. In one embodiment, the viscosity may be adjusted to a range of about 0.8 to about 3 mPas. In another embodiment, the dynamic dynamic viscosity of the composition is in the range of about 0.8 to about 2.5 mPas or even about 0.9 to about 1.3 mPas at ambient temperature.

Typically, the cyclodextrin concentration in the composition according to the present invention is in the range from about 1 to about 100 mg/ml, preferably from 5 to about 50 mg/ml, and more preferably from about 10 to about 30 mg/ml. Other preferred concentrations range from about 15 to about 25 mg/ml.

The present inventors have found that the administration of cyclodextrins may directly treat or prevent nasal inflammations. In particular, the present inventors have found that the topical application of cyclodextrins to the nasal mucosa treats or prevents nasal inflammations. In particular, the present inventors have found that this is the direct effect of the topical administration of cyclodextrins to the nose. Whilst other compounds may be present, there is no need for administering a further active pharmaceutical ingredient and in particular a further anti-inflammatory active pharmaceutical ingredient to obtain a positive effect in respect of nasal inflammations.

Accordingly, in one embodiment, no further active pharmaceutical ingredient may be present in the composition of the present invention, wherein cyclodextrins are primarily used as solubilizing excipient or as a drug delivery system. In another embodiment, no one or a combination of following pharmaceutically active compounds is present: non-steroidal anti-inflammatory drugs (NSAIDs), leukotriene-antagonists such as roflumilast; or betasympathomimetics, anticholinergics, local anesthetics, immunomodulators, antiinfectives, angiotensin converting enzyme (ACE) inhibitors, cytostatics, formoterol, salmeterol, levalbuterol; tiotropium, oxitropium, ipratropium; lidocaine, prolocaine, mepivacaine, bupivacaine, articaine, ciclosporine, tacrolimus, sirolimus, everolimus, rapamycin, azathioprine; ciprofloxacine, moxifloxacine, azithromycine, clarithromycine, erythromycine, metronidazole, ketoconazole, itraconazole, voriconazole, clotrimazole, bifonazole, fluconazole, amphotericine B, natamycine, nystatine, amikacine, acyclovir, famciclovir, valaciclovir, didanosine, saquinavir, ritonavir, lamivudine, stavudine, zidovudine, ribivarine, captoprile, lisinoprile, perindoprile, trandolaprile, cilazaprile, carmustine, lomustine, taxol, etoposide, cisplatin; reduced glutathione, TNF-alpha-antibodies including the pharmaceutically acceptable derivatives of these substances such as, for example, the salts, conjugates, enantiomeres, racemates, epimeres, diastereomeres or complexes of any one of these active pharmaceutical ingredients.

In one embodiment, the composition of the invention is aqueous, which means that it is preferably a liquid composition comprising water as the predominant liquid constituent. As the composition is intended for administration in aerosol form, it is further preferred for safety and tolerability reasons that water is the only liquid present in the composition. However, in some cases it may be acceptable even for liquids for inhalation to comprise some amount of other liquids, in particular one or more organic solvents having a relatively low inhalation toxicity such as ethanol, propylene glycol, or glycerol. Typically, these liquids are present in very low amounts, typically in the range of 0.1 to 5 mg/ml.

In a further embodiment of the invention, the inhalable composition further comprises a component selected from the group consisting of carriers, stabilizers, diluents, dispersing agents, suspending agents, thickening agents, excipients, and antimicrobial preservatives. Typically, these components are present in very low amounts, typically in the range of 0.1 to 5 mg/ml.

The aqueous composition is suitable for administration as an aerosol. In particular, it is designed for nasal inhalation for the prevention or treatment of a symptom or disease affecting a region of the upper respiratory tract, such as the nasal mucosa or the paranasal sinuses.

The cyclodextrins may be administered in an isotonic solution or a hypertonic solution.

A solution is isotonic when its effective osmole concentration is the same as that of the cytosol inside the cell and in particular the nasal mucosa cells.

A hypertonic solution is called hypertonic if it has a greater concentration of solutes than the cytosol inside the cell.

It is further preferred that the pH of the composition is adjusted to 3.5 to 7.5, preferably from 6.5 to 7.

In order to adjust the pH, surface tension, viscosity, osmolality, stability, taste and other properties of the composition, one or more further excipients may be used. For example, the composition may comprise one or more excipients selected from pharmaceutically acceptable organic acids, salts of organic acids, inorganic acids, inorganic salts, bases, sugars, sugar alcohols, stabilizers, antioxidants, surfactants, preservatives, and taste masking agents.

A further object of the invention is a composition consisting of:
a) 1 to 100 mg/ml, preferably, 5 to 50 mg/ml, even more preferably 10 to 30 mg/ml of one or more cyclodextrins or pharmaceutically acceptable derivatives thereof, in an alternative embodiment the concentration of cyclodextrins is 50 millimolar to 100 millimolar, preferably 10 millimolar to 80 millimolar, even more preferably 20 millimolar to 70 millimolar, even more preferably 40 millimolar to 60 millimolar, even more preferably 45 millimolar to 55 millimolar,
b) 0.1 to 5 mg/ml, preferably, 0.5 to 3 mg/ml, more preferably, 1 to 3 mg/ml of a pharmaceutically acceptable salt selected from the group consisting of pH buffers, sea salt, and salt and
c) water.

The composition of the present invention enables aqueous and dry powder formulations whose properties allow highly efficient and convenient aerosol or dry powder delivery using currently available aerosol generating devices, and in particular nasal aerosolization devices or dry powder inhalers.

Accordingly, a further object of the invention is the use of the composition of the invention in the prevention or treatment of nasal inflammations.

Aerosolization, in the sense of the present invention, means any spraying process that produces droplets. The size of the droplets may vary. Typically, the size of the droplets is between 1 and 100 microns, preferably, between 1.5 and 10 microns and even more preferably between 2 and 7 microns.

Micronization, in the sense of the present invention, means any process to decrease the particle size of dry powders to an inhalable size, preferably between 1 and 100 microns, preferably, between 1.5 and 10 microns and even more preferably between 2 and 7 microns.

Dry powder inhaler, in the sense of the present invention, means any device suitable for micronization of inhalable dry powder compositions and in particular for nasal inhalation.

Finally, the composition of the present invention may have very little side effects even when administered even at very high dosages, such as daily delivered doses up to 180 mg/kg, as shown in example 1.

In one embodiment of the invention, the composition reduces the number of goblet cells in nasal mucosa tissue, for example after exposure to ovalbumin by more than 20% in sensitized animals, as shown in Example 2 and FIG. 1. Goblet cells are simple columnar epithelial cells that secrete gel-forming mucins. Goblet cells are typically found in the respiratory tracts. Differentiation of epithelial cells into goblet cells plays a key role in the excessive mucus production. Thus, the number of goblet cells in nasal mucosa tissue is indicative of the state of the inflammation.

### DRAWINGS

FIG. 1 shows histopathology at 4 levels of the nasal cavity of Wistar rats of FIG. 1 exposed to low, middle and high doses of HPBCD.

FIG. 2 shows the effect of the administration of cyclodextrins to Wistar rat nasal goblet cells after exposure to Ovalbumin.

### EXAMPLES

### Example 1—Safety

Hydroxypropylbetacyclodextrin (HPBCD) was administered during two-weeks in a Dose-Range-Finding (DRF) Inhalation Study to Wistar rats in three different delivered doses:
low of 20 mg/kg/day and a target atmosphere concentration of 0.3 mg/L,
medium of 60 mg/kg/day and a target atmosphere concentration of 0.9 mg/L, and
concentration of 2.7 mg/L to each three male and three non-pregnant female rats.

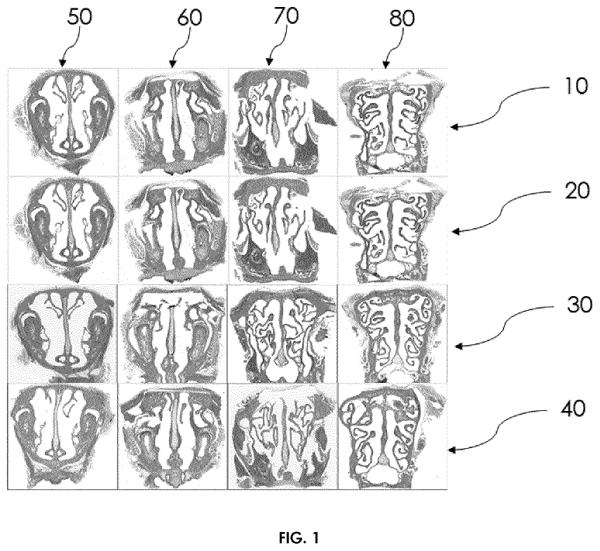

A control group which received control vehicle in the form of citrate buffer was provided with three male and three non-pregnant female rats. After terminal euthanasia, the nasal cavity was removed. Nasal cavities were embedded in paraffin wax, sections were cut at 4-6 μm by microtome and transferred to slides. Tissue sections were stained with haematoxylin-eosin/phloxine and examined by light microscope. Full histopathology of the nasal cavities, as well as the other organs of the respiratory tract, was performed in each group and is shown in FIG. 1. 4 levels of the nasal cavities were examined, among which 1 level included the nasopharyngeal duct and Nasal Associated Lymphoid Tissue (NALT).

FIG. 1 shows the control group (line 10) and the low (line 20), mid (line 30) and high (line 40) at four different section levels represented in columns 50, 60, 70 and 80. Nasal cavities analyzed by histopathology were normal at all exposure levels of HPBCD.

Table 1 shows the design of the Safety study of example 1 and table 2 shows its results.

TABLE 1
Design of Safety study

| | | | Achieved | | | | |
|---|---|---|---|---|---|---|---|
| | | | HPBCD | Nose-only expose | | | |
| | | Target | doses | | Total | | |
| | | HPBCD | based on | Duration | duration | | |
| | HPSCD Target | atmosphere | gravimetry | of daily | of | Animal | |
| | Delivered Dose | concentration | (mg/kg/day) | exposure | exposure | number/group | |
| Dosage | (mg/kg/day) | (mg/L) | n = 14 | (min) | (days) | Male | Female |
| Control | 0 | 0 | 0 | 90 | 14 | 3 | 3 |
| Low | 20 | 0.3 | 22.17 | 90 | 14 | 3 | 3 |
| Mid | 60 | 0.9 | 92.04 | 90 | 14 | 3 | 3 |
| High | 180 | 2.7 | 190.80 | 90 | 14 | 3 | 3 |

TABLE 2
Results of the Safety study

| | Male | | | | Female | | | |
|---|---|---|---|---|---|---|---|---|
| | Dosage | | | | | | | |
| | Control | Low | Mid | High | Control | Low | Mid | High |
| Animals | 3 | 3 | 3 | 3 | 3 | 3 | 3 | 3 |
| Submitted | 3 | 3 | 3 | 3 | 3 | 3 | 3 | 3 |
| Examined | 3 | 3 | 3 | 3 | 3 | 3 | 3 | 3 |
| Normal | 3 | 3 | 3 | 3 | 3 | 3 | 3 | 3 |

### Example 2—Efficacy

Eight male and female BALB/c mice (6-8 weeks of age at the beginning of the protocol) were sensitized with Ovalbulmin (OVA) on D1 and D11 by intraperitoneal (i.p.) injection (10 μg grade V OVA diluted in 200 μl PBS), followed by OVA aerosols (10% grade Ill OVA dissolved in PBS buffer −50 ml were aerosolized with ultrasonic nebulizer during 30 minutes in a 30×20×15 cm Plexiglas exposure box where the animals were authorized to breathe and move normally). Aerosol administration was performed 360 min after i.n. treatments with Placebo or hydroxypropyl-gamma-cyclodextrin (HP-gamma-CD) during even weeks from days 22 to 98 (5 days/even week).

Mice were treated from Day 22 to Day 98 (5 days per week even week) with PBS (8 mice in control group) or with hydroxypropyl-gamma-cyclodextrin 50 mM diluted in PBS (8 mice in treated group) by aeorosolization (50 ml were aerosolized during 30 minutes in a Plexiglas exposure box where the animals were authorized to breathe and move normally) 360 min before each OVA inhalation.

On Day 99, the animals were sacrificed. The nasal cavity was removed, fixed with 4% paraformaldehyde and treated with decalcifier DC2 during 24 h. Nasal cavities were then embedded in paraffin wax and sections were cut at 5 micrometers by microtome and transferred to slides. Tissue sections were stained with haematoxylin-eosin and Periodic Acid Schiff (PAS) and scanned with a Hamamatsu Nanozoomer HT 2.0 at a 40× magnification.

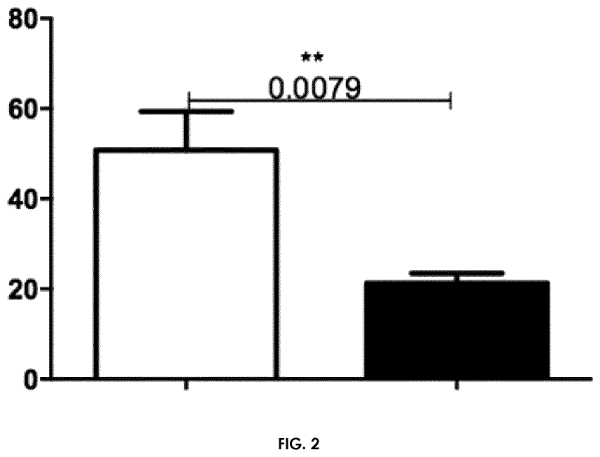

Numbers of goblet cells were counted using digitalized images of histological slides. Three hundred epithelial cells were counted at random and the percentage of positive cells for PAS staining (Goblet cells) was established relative to cells negative for PAS staining. Results are shown in FIG. 2.

FIG. 2 shows the reduction of goblet cells through administration of Hydroxypropyl-gamma-cyclodextrin after exposure to Ovalbumin in nasal mucosa tissue of mice. The % of goblet cells in nasal mucosa tissue of mice is shown on the vertical axis. Hydroxypropyl-gamma-cyclodextrin as shown in the second black column leads to a reduction from 50% (control group, white column) to 20% of goblet cells.

Whilst the cyclodextrin concentration for mice was chosen at 50 mM, concentrations for human use may be chosen, for example from 0.5 mM to 100 mM, preferably 1 mM to 75 mM, even more preferably 5 mM to 50 mM, even more preferably 10 mM to 40 mM, even more preferably 20 mM to 30 mM and even more preferably 25 mM.

The present invention is not limited to the described embodiments and that variations can be applied without going outside of the scope of the appended claims.

1. Inhalable composition comprising one or more cyclodextrins or pharmaceutically acceptable derivatives thereof for use in the prevention or treatment of nasal inflammations, wherein the treatment or prevention of nasal inflammations is a topical treatment.

2. Inhalable composition according to claim 1, wherein the nasal inflammation is a viral nasal inflammation.

3. Inhalable composition claim 1, wherein:
a) no further active pharmaceutical ingredient is present in therapeutically effective amounts for which cyclodextrin is used to deliver an active pharmaceutical ingredient to other parts of the body than the nose or the nasal mucosa;
b) no further active pharmaceutical ingredient is present in therapeutically effective amounts for which cyclodextrin is used to increase its solubility in water or
c) no further anti-inflammatory active pharmaceutical ingredient is present in therapeutically effective amounts.

4. Inhalable composition claim 1 wherein the inhalable composition is an aerosolizable aqueous composition, a topical nasal spray or an inhalable dry powder composition.

5. Inhalable composition of claim 1, wherein the pH of the aqueous composition is from 6.5 to 7.

6. Inhalable composition according to claim 1, wherein the cyclodextrin is selected from the group consisting of alpha-cyclodextrin, beta-cyclodextrins, γ-cyclodextrin, 2-hydroxypropyl-beta-cyclodextrin, 2-hydroxypropyl-γ-cyclodextrin, sulfobutylether-beta-cyclodextrin, and methyl-beta-cyclodextrin.

7. Inhalable composition claim 1, wherein the cyclodextrin is hydroxypropylbetacyclo-dextrin.

8. Inhalable composition according claim 1, wherein no corticosteroid or oil is present in effective amounts.

9. Inhalable composition according to claim 1, wherein the inhalable composition is isotonic or hypertonic.

10. Inhalable composition according to claim 1, wherein the concentration of cyclodextrins is from 0.5 mM to 100 mM.

11. Inhalable composition according to claim 1, wherein the concentration of cyclodextrins is from 20 mM to 30 mM.

12. Composition of claim 1, wherein the composition consists of:
a) 0.5 mM to 100 mM of one or more cyclodextrins or pharmaceutically acceptable derivatives thereof;
b) 0.1 to 5 mg/ml of a pharmaceutically acceptable salt selected from the group consisting of pH buffers, sea salt, and salt and
c) water.

13. Inhalable composition according to claim 1, wherein the composition consists of:
a) 10 mM to 40 mM of one or more cyclodextrins or pharmaceutically acceptable derivatives thereof;
b) 0.5 to 3 mg/ml of a pharmaceutically acceptable salt selected from the group consisting of pH buffers, sea salt, and salt and
c) water.

14. Inhalable composition according to claim 1, wherein the composition consists of:
a) 20 to 30 mM of one or more cyclodextrins or pharmaceutically acceptable derivatives thereof;
b) 0.5 to 3 mg/ml of a pharmaceutically acceptable salt selected from the group consisting of pH buffers, sea salt, and salt and
c) water.

15. Aerosol-generating device or dry powder inhaler comprising a composition according to claim 1, for use in the prevention or treatment of nasal inflammations.
